(19) United States
(12) Patent Application Publication
(10) Pub. No.: US 2009/0191609 A1
(43) Pub. Date: Jul. 30, 2009

(54) Compositions and methods for enhancing paper product degradation

(76) Inventors: Andrew R. Lefkowitz, University Heights, OH (US); Marc-David Bismuth, Los Angeles, CA (US); Sean Farmer, Miami Beach, FL (US)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 12/229,842

(22) Filed: Aug. 27, 2008

Related U.S. Application Data
(60) Provisional application No. 60/966,869, filed on Aug. 29, 2007.

(51) Int. Cl. C12N 1/20

(52) U.S. Cl. 435/252.5

(57) ABSTRACT

The present invention describes compositions and methods for enhancing biodegradation of paper products after use and disposal.

### FIELD OF THE INVENTION

The invention relates to the use of lactic acid-producing bacteria to enhance the biodegradability of paper products.

### BACKGROUND

Disposal of paper products is a major environmental concern due to the large volume of disposed material. Paper products such as paper grocery bags, paper plates, newspapers and the like biodegrade slowly and occupy considerable space due to the bulk of these products. As such, landfills are overused and accumulate excessive amounts of disposed paper products. Thus, there is a pressing need for improvements in biodegradation of paper products.

### SUMMARY OF THE INVENTION

The invention describes the use of acid-producing, heterotrophic bacteria to enhance the degradation of paper products. Bacillus coagulans bacteria are included in the compositions and methods of this invention and are referred to herein as “degradative bacteria”.

The invention provides for articles of manufacture including a paper composition and an isolated Bacillus coagulans bacterium. In one aspect, the paper composition includes cellulose. Suitable types of cellulose include wood cellulose, cotton cellulose, linen cellulose, grass cellulose, rice cellulose, and hemp cellulose. Optionally, the wood cellulose comprises wood pulp of a pine, spruce, cedar, fir, hemlock, larch, cypress, yew, aspen, eucalyptus, or birch tree. An exemplary paper composition includes a paper plate, a paper bag, a cardboard container, a textile, a newspaper, a writing paper or a napkin. Preferably, the paper composition is a paper grocery bag.

The rate of biodegradation of the paper composition in the presence of the isolated Bacillus coagulans bacterium is at least 10%, at least 25%, at least 50%, at least 75%, or at least 100% greater than the rate of biodegradation in the absence of said isolated Bacillus coagulans bacterium. In one aspect, the isolated Bacillus coagulans are between 1% and 10% by weight of the article of manufacture.

Bacterial species include Bacillus coagulans, e.g., Bacillus coagulans hammer, preferably Bacillus coagulans hammer strain Accession No. ATCC 31284, or one or more strains derived from Bacillus coagulans hammer strain Accession No. ATCC 31284 (e.g. ATCC Numbers: GBI-20, ATCC Designation Number PTA-6085; GBI-30, ATCC Designation Number PTA-6086; and GBI-40, ATCC Designation Number PTA-6087; see U.S. Pat. No. 6,849,256 to Farmer).

Optionally, the isolated Bacillus coagulans is in the form of a spore. Alternatively, the isolated Bacillus coagulans is in the form of a vegetative cell. In yet another aspect, the isolated Bacillus coagulans is in the form of a mixture of vegetative cells and spores.

The invention also provides for methods of increasing the biodegradation rate of a paper composition by applying an isolated Bacillus coagulans bacterium to the paper composition. Preferably, the paper composition is a paper grocery bag.

Optionally, the isolated Bacillus coagulans bacterium is applied prior to or during a stage of manufacture of the paper composition. In one aspect, the isolated Bacillus coagulans is introduced into a paper paste or paper pulp, such as wood pulp. Suitable wood pulp includes pulp from pine, spruce, cedar, fir, hemlock, larch, cypress, yew, aspen, eucalyptus, or birch trees.

Alternatively, the isolated Bacillus coagulans is applied onto the surface of a finished paper product after the manufacture of the paper product has concluded. Preferably, the isolated Bacillus coagulans is spray-dried onto the finished paper product.

The invention also provides for methods for increasing the biodegradation rate of paper compositions, wherein the rate of biodegradation of the paper composition in the presence of the isolated Bacillus coagulans is at least 10%, at least 25%, at least 50%, at least 75%, or at least 100% greater than the rate of biodegradation in the absence of the isolated Bacillus coagulans bacterium.

Cited publications are incorporated herein by reference. Both the foregoing general description and the following detailed description and examples are exemplary and explanatory only and are not restrictive of the invention as claimed.

### DETAILED DESCRIPTION

The present invention is directed to the discovery that non-pathogenic lactic acid-producing bacteria (i.e., “lactic acid bacteria”), such as the exemplary Bacillus coagulans, are useful in compositions to enhance the biodegradability of paper products.

### Probiotic Lactic Acid-Producing Bacteria

A probiotic lactic acid-producing bacteria suitable for use in the methods and compositions of the invention produces acid and is non-pathogenic. There are many suitable bacteria identified as described herein, although the invention is not limited to currently known bacterial species insofar as the purposes and objectives of the bacteria is described. The property of acid production is important to the effectiveness of the probiotic lactic acid-producing bacteria of this invention.

The invention provides using a lactic acid-producing bacteria, such as a spore-forming Bacillus species, such as B. coagulans. Preferably, the spore-forming Bacillus species of the invention is B. coagulans Hammer. Purified or isolated Bacillus coagulans is particularly useful in the present invention. B. coagulans is non-pathogenic and is generally regarded as safe (i.e., GRAS classification) by the U.S. Federal Drug Administration (FDA) and the U.S. Department of Agriculture (USDA), and by those skilled in the art.

Bacillus coagulans is a non-pathogenic gram positive spore-forming bacteria that produces L(+) lactic acid (dextrorotatory) in fermentation conditions. It has been isolated from natural sources, such as heat-treated soil samples inoculated into nutrient medium (Bergey's Manual off Systemic Bacteriology, Vol. 2, Sneath, P. H. A., et al., eds., Williams & Wilkins, Baltimore, Md., 1986). Bacterial enzymes and other metabolic products produced by probiotic lactic acid-producing bacteria play an important role in the biodegradation of many paper products. Purified B. coagulans strains have served as a source of enzymes including endonucleases (e.g., U.S. Pat. No. 5,200,336); amylase (U.S. Pat. No. 4,980,180); lactase (U.S. Pat. No. 4,323,651); and cyclo-malto-dextrin glucano-transferase (U.S. Pat. No. 5,102,800). B. coagulans has been used to produce lactic acid (U.S. Pat. No. 5,079,164). A strain of B. coagulans (referred to as L. sporogenes; Sakaguti & Nakayama (ATCC 31284)) has been combined with other lactic acid producing bacteria and B. natto to produce a fermented food product from steamed soybeans (U.S. Pat. No. 4,110,477).

Bacterial species include Bacillus coagulans, e.g., Bacillus coagulans hammer, preferably Bacillus coagulans hammer strain Accession No. ATCC 31284, or one or more strains derived from Bacillus coagulans hammer strain Accession No. ATCC 31284 (e.g., ATCC Numbers: GBI-20, ATCC Designation Number PTA-6085; GBI-30, ATCC Designation Number PTA-6086; and GBI-40, ATCC Designation Number PTA-6087; see U.S. Pat. No. 6,849,256 to Farmer).

In one aspect, the Bacillus coagulans bacteria of the invention are included in the composition in the form of vegetative cells. Alternatively, the Bacillus coagulans bacterium is included in the composition in the form of spores. In another aspect, a Bacillus coagulans strain is included in the composition in the form of a dried cell mass, a stabilized paste, or a stabilized gel.

Because Bacillus spores are heat-resistant and additionally can be stored as a dry power, they are particularly useful for formulation into and manufacture of dry products such as the various paper products and compositions of the invention. Heat and pressure-resistant spores are also suitable for use in pressure-treated paper compositions of the invention. Bacillus species are particularly suited for the present invention, particularly species having the ability to form spores which are relatively resistant to heat and other conditions, making them ideal for storage (shelf-life) in product formulations.

### Paper Compositions

The present invention is directed to the discovery that lactic acid-producing bacteria, particularly Bacillus species, are used in compositions to enhance the biodegradability of paper products. As discussed further, the compositions can be formulated in many configurations because the bacterium can be presented as a vegetative cell or as a spore, or both, depending on the species and form of the probiotic organism. The cells/spores can be presented in a variety of compositions suited for use in a paper composition.

Exemplary paper compositions include paper plates, paper bags, cardboard containers, textiles, newspapers, writing papers or napkins. Preferably, the paper composition is a paper grocery bag. Optionally, the paper composition includes cellulose, such as wood cellulose, cotton cellulose, linen cellulose, grass cellulose, or hemp cellulose. Suitable wood cellulose includes the wood pulp of pine, spruce, cedar, fir, hemlock, larch, cypress, yew, aspen, eucalyptus, and birch trees.

In one aspect, the Bacillus bacterium and/or the isolated active agent is impregnated into the paper product during the manufacturing process of the paper product (e.g., added to a synthetic composition before or during the polymerization process). The pressure and heat resistance of Bacillus spores makes them particularly suitable for incorporation into the material during manufacturing. The finished paper composition is stored in a package prior to use or is used immediately.

Alternatively, the Bacillus bacterium and/or Bacillus coagulans isolated active agent is applied to a finished paper product using any of a variety of known methods including, for example, applying a powder, spray-drying the probiotic onto the paper product or soaking the paper product in a solution containing the probiotic and then using the wetted paper product or drying the paper product prior to use. Porous paper products may contain the Bacillus and/or the isolated active agent in the pores or interstices of the paper product.

Those skilled in the art will recognize that any of a variety of methods for placing the bacterial composition onto a paper product can be used. However, preferred methods include a “spray-dry” method in which the paper product is exposed in a low humidity chamber to an atomized mix containing a liquid composition, where the chamber is subsequently exposed to approximately 80-110° F. to dry the liquid, thereby impregnating the material of the paper product with the components of the composition.

A typical concentration is from approximately 1×10⁵ to 1×10¹⁰ CFU of viable bacterium or spores/in² of external surface of paper product; 1×10⁶ to 1×10⁹ CFU of viable bacterium or spores/in² of external surface of paper product; or 1×10⁷ to 1×10⁸ CFU of viable bacterium or spores/in² of external surface of paper product. Following drying, the paper product is ready for storage in a package, or for direct use.

Preferably, the probiotic lactic acid-producing bacteria is introduced into or onto portions of the paper composition by applying a composition containing viable bacteria to the paper composition during a stage of the manufacture of the paper product. In one aspect, the spores and/or vegetative cells of the probiotic acid-producing bacteria are introduced into the paper paste or pulp during a stage of the manufacture of the paper product. Optionally, the paper pulp is wood pulp.

The invention describes that the active ingredients (i.e., live bacteria or extracellular components) comprise about 0.1% to about 50% by weight of the final composition, preferably 1% to 10% by weight of the final composition. The invention provides paper compositions that include a degradation-enhancing bacteria as described herein. A preferred amount of this bacteria is an amount sufficient to promote degradation, which is from about 10⁴ to 10¹⁴ CFU of bacteria (i.e., vegetative cells and/or bacterial spores) for use per unit of paper product, preferably about 10⁷ to 10¹⁰ CFU per unit, and more preferably about 10⁸ to 10⁹ CFU per unit. The actual amount of bacteria in a paper composition will vary depending upon the amounts of composition to be dispersed into the paper composition and upon routes of dispersal.

In another aspect, the invention provides methods of increasing the biodegradation rate of paper products by applying an isolated Bacillus coagulans bacterium to the paper composition. Biodegradation is the process by which organic substances are broken down by other living organisms. The rate of biodegradation of the paper product in the presence of Bacillus coagulans is at least 10%, at least 25%, at least 50%, at least 75% or at least 100% greater than the rate of biodegradation in the absence of Bacillus coagulans bacterium. Alternatively, the rate of biodegradation of said paper product in the presence of Bacillus coagulans is at least two-fold, at least four-fold, at least six-fold, at least eight-fold, or at least ten-fold greater than the rate of biodegradation in the absence of Bacillus coagulans bacterium.

The present invention provides for an increase in the rate of biodegradation of paper products under a variety of environmental conditions. In one aspect, the rate of biodegradation is increased under conditions of standard temperature and pressure, i.e. 273.15 degrees Kelvin (zero degrees Celsius) and 760 mmHg (1 atmosphere). In another aspect, the rate of biodegradation is increased at temperatures between 0° C. and 100° C., such as 25° C., 35° C., 50° C., and 75° C. In yet another aspect, the rate of biodegradation is increased at pressures between 1 atmosphere (atm) and 10 atm. Optionally, the rate of biodegradation is increased in the presence or absence of direct or indirect sunlight or artificial light.

### EXAMPLE 1

### Preparation of Bacillus coagulans Cultures

Bacillus coagulans Hammer bacteria (ATCC Accession No. 31284) was inoculated and grown to a cell density of about 10⁸ to 10⁹ cells/ml in nutrient broth containing 5 g Peptone, 3 g Meat extract, 10-30 mg MnSO₄, and 1,000 ml distilled water, adjusted to pH 7.0, using a standard airlift fermentation vessel at 30° C. The range of MnSO₄ acceptable for sporulation is 1 mg/l to 1 g/l. The vegetative cells can actively reproduce up to 45° C., and the spores are stable up to 90° C. After fermentation, the B. coagulans bacterial cells or spores are collected using standard methods (e.g., filtration, centrifugation) and the collected cells and spores can be lyophilized, spray-dried, air-dried or frozen. As described herein, the supernatant from the cell culture is collected and used as an extracellular agent secreted by B. coagulans.

A typical yield from the above culture is in the range of about 10⁹ to 10¹⁰ viable spores and more typically about 100 to 150 billion cells/spores per gram before drying. Spores maintain at least 90% viability after drying when stored at room temperature for up to ten years, and thus the effective shelf life of a composition containing B. coagulans Hammer spores at room temperature is about 10 years.

### EXAMPLE 2

### Preparation of Bacillus coagulans Spores

A culture of dried B. coagulans spores was alternately prepared as follows. Ten million spores were inoculated into a one liter culture containing 24 g potato dextrose broth, 10 g of enzymic-digest of poultry and fish tissue, 5 g of FOS and 10 g MnSO4. The culture was maintained for 72 hours under a high oxygen environment at 37° C. to produce culture having about 150 billion cells per gram of culture. Thereafter, the culture was filtered to remove culture medium liquid, and the bacterial pellet was resuspended in water and freeze-dried. The freeze-dried powder is then ground to a fine powder using standard good manufacturing practice (GMP).

What is claimed is:

1. An article of manufacture comprising a paper composition and an isolated Bacillus coagulans bacterium.

2. The article of manufacture of claim 1, wherein said paper composition comprises cellulose, wherein said cellulose is selected from the group consisting of wood cellulose, cotton cellulose, linen cellulose, grass cellulose, rice cellulose, and hemp cellulose.

3. The article of manufacture of claim 2, wherein said wood cellulose comprises wood pulp of a tree selected from the group consisting of pine, spruce, cedar, fir, hemlock, larch, cypress, yew, aspen, eucalyptus, and birch.

4. The article of manufacture of claim 1, wherein said paper composition is selected from the group consisting of a paper plate, a paper bag, a cardboard container, a textile, a newspaper, a writing paper or a napkin.

5. The article of manufacture of claim 4, wherein said paper bag is a paper grocery bag.

6. The article of manufacture of claim 1, wherein the rate of biodegradation of said paper composition in the presence of said isolated Bacillus coagulans bacterium is at least 10% greater than the rate of biodegradation in the absence of said isolated Bacillus coagulans bacterium.

7. The article of manufacture of claim 1, wherein said isolated Bacillus coagulans comprises between 1% and 10% by weight of said article of manufacture.

8. The article of manufacture of claim 1, wherein said isolated Bacillus coagulans is GBI-30 strain (ATCC Designation Number PTA-6086).

9. The article of manufacture of claim 1, wherein said isolated Bacillus coagulans is GBI-20 strain (ATCC Designation Number PTA-6085).

10. The article of manufacture of claim 1, wherein said isolated Bacillus coagulans is GBI-40 strain (ATCC Designation Number PTA-6087).

11. The article of manufacture of claim 1, wherein said isolated Bacillus coagulans is in the form of a spore.

12. The article of manufacture of claim 1, wherein said isolated Bacillus coagulans is in the form of a vegetative cell.

13. A method for increasing the biodegradation rate of a paper composition comprising applying an isolated Bacillus coagulans bacterium to said paper composition.

14. The method of claim 13, wherein said applying occurs during a stage of manufacture of said paper composition.

15. The method of claim 14, wherein said isolated Bacillus coagulans is introduced into a paper paste or paper pulp.

16. The method of claim 15, wherein said paper pulp is wood pulp.

17. The method of claim 13, wherein said isolated Bacillus coagulans is applied onto the surface of a finished paper product.

18. The method of claim 17, wherein said isolated Bacillus coagulans is spray-dried onto said finished paper product.

19. The method of claim 13, wherein the rate of biodegradation of said paper composition in the presence of said isolated Bacillus coagulans is at least 10% greater than the rate of biodegradation in the absence of said isolated Bacillus coagulans bacterium.

20. The method of claim 13, wherein said paper composition is a paper grocery bag.
